(19) United States
(12) Patent Application Publication
(10) Pub. No.: US 2024/0358265 A1
(43) Pub. Date: Oct. 31, 2024

(54) METHOD AND PROGRAM PRODUCT FOR DETERMINING PERIODIC SIGNAL, METHOD AND DEVICE FOR DETERMINING PPG SIGNAL

(71) Applicant: Wistron Corp., New Taipei City (TW)

(72) Inventors: Ming-Chiuan JING, New Taipei City (TW); Hsuan Tsung CHANG, New Taipei City (TW); Chin-Hao HSU, New Taipei City (TW)

(21) Appl. No.: 18/452,701

(22) Filed: Aug. 21, 2023

(30) Foreign Application Priority Data
Apr. 26, 2023 (TW) 112115448

(51) Int. Cl. A61B 5/024; G16H 50/70

(52) U.S. Cl. CPC A61B 5/02416; G16H 50/70

(57) ABSTRACT

A method for determining whether a signal is periodic is provided and performed by a processing device. In the method, a signal S is sampled at a time interval t to obtain (k+1) signal values S(t0+i·t), wherein i=0˜k,A 2D dot plot on an XY plane is generated, wherein the coordinates (x, y) of each point in the two-dimensional dot plot are (S_{PPG} (10+i·t), S_{PPG} (t0+(1+1)·t), wherein i=0˜(k−1); The degree of dispersion of the 2D dot plot is calculated. The relative relationship between the degree of dispersion and a feature of a periodic signal is determined and a determination result is generated.

### BACKGROUND OF THE INVENTION

### Cross Reference to Related Applications

This application claims priority of Taiwan Patent Application No. 112115448, filed on Apr. 26, 2023, the entirety of which is incorporated by reference herein.

### FIELD OF THE INVENTION

The present disclosure relates to method and program products for determining whether a signal is periodic, and method and device for identifying photoplethysmography (PPG) signal.

### DESCRIPTION OF THE RELATED ART

When processing signals, if an input signal is defective, the output result will be affected such that the output signal will also be defective, and may even be unreliable. To obtain reliable results, it is necessary to filter the input signal.

In the biomedical field, biomedical signals that are being measured often contain severe irregular noises, due to the pollution of the detection environment or an undesirable posture or angle. Because most biomedical signals are periodic signals and will become non-periodic signals after being mixed with irregular noises, a determination can be made as to whether the input signal is defective by determining whether the input signal is periodic, so as to filter the input signal.

Existing techniques to distinguish whether a signal is periodic or not usually include a waveform analysis or Fourier transform. However, waveform analysis requires the positions of wave crests and wave troughs to find the waveform of each cycle for comparison, and the located position may be wrong. Thus, waveform analysis is not only computationally intensive, but also inaccurate. Moreover, Fourier computation also requires huge and complex computations. The reliability of the results of a Fourier characteristic analysis is not ideal when a periodic signal contains noise.

Due to the huge amount of computation required, the methods mentioned above require a long time to complete, even with a powerful processor, and the power consumption and the size of the computing device are significant. However, when measuring biomedical signals, it is often necessary to operate for a long time and to carry the measurement device. Thus, a less computationally intensive method for filtering the signals is necessary. The method can be performed by a small processor with low power consumption to determine whether the input signal is a periodic signal.

### BRIEF SUMMARY OF THE INVENTION

Embodiments of the present disclosure provide a method for determining whether a signal is periodic. The method can be configured to determine whether a signal is a periodic signal with a small amount of calculation.

In an embodiment of the present disclosure, a method for determining whether a signal is periodic is provided. The method may be performed by a processing device and includes: sampling a signal S at a time interval t to obtain (k+1) signal values S(t0+i·t), wherein i=0˜k; generating a two-dimensional dot plot on an XY coordinate plane, wherein the coordinates (x, y) of each point in the two-dimensional dot plot are (S(t0+i·t), S(t0+(i+1) t)), wherein i=0˜(k−1); calculating a degree of dispersion of the two-dimensional dot plot; determining a relative relationship between the degree of dispersion and a feature of the periodic signal and generating a determination result; and outputting the determination result.

In an embodiment of the present disclosure, a photoplethysmography (PPG) signal analysis device is provided. The PPG signal analysis device comprises a PPG signal input device, a memory device, and a processing device. The PPG signal input device is configured to obtain a PPG signal S_{PPG}. The memory device is configured to store an application. The processing device is configured to run the application to implement the functions of the following sections: a sampling part, a drawing part, a calculation part, a determination part, and an output part. The sampling part is configured to sample the photoplethysmography signal S_{PPG} at the time interval t to obtain (k+1) signal values S_{PPG} (t0+i·t), wherein i=0˜k. The drawing part is configured to generate a two-dimensional dot plot on an XY coordinate plane. The coordinates (x, y) of each point in the two-dimensional dot plot are (S_{PPG} (t0+i·t), S_{PPG} (t0+(i+1)·t)), wherein i=0˜(k−1). The calculation part is configured to calculate the degree of dispersion of the two-dimensional dot plot. The determination part is configured to determine a relative relationship between the degree of dispersion and a feature of a periodic signal and to generate a determination result. The output part is configured to output an alert message in response to the determination result. The alert message indicates that the relative relationship between the degree of dispersion and the feature of the periodic signal does not comply with the predefined standard.

### BRIEF DESCRIPTION OF THE DRAWINGS

The present disclosure can be more fully understood by reading the subsequent detailed description and examples with references made to the accompanying drawings, wherein:

FIG. 1 shows the flow charts of the embodiment 1 and the embodiment 1A of the present disclosure;

FIG. 2A is a schematic diagram of the periodic signal P in accordance with embodiment 1 of the present disclosure;

FIG. 2B is a two-dimensional (2D) dot plot corresponding to the periodic signal P;

FIG. 2C is a schematic diagram of a regression line of the 2D dot plot of the periodic signal P;

FIG. 3A is a schematic diagram of non-periodic signal (noise) N in accordance with embodiment 1 of the present disclosure;

FIG. 3B is a 2D dot plot corresponding to the non-periodic signal (noise) N;

FIG. 3C is a schematic diagram of a regression line of the 2D dot plot of the non-periodic signal (noise) N;

FIG. 4 is a schematic diagram of the projection distance of the least squares method.

FIG. 5 is a schematic diagram of k-means method in accordance with embodiment 1 of the present disclosure;

FIG. 6 is a template diagram in accordance with embodiment 1A of the present disclosure;

FIG. 7A shows an example of the sampled periodic signal in accordance with embodiment 1 of the present disclosure;

FIGS. 7B˜7G are 2D dot plots drawn by sampling the periodic signal of FIG. 7A with different time intervals;

FIG. 8A shows an example of the sampled non-periodic signal in accordance with embodiment 1A of the present disclosure;

FIGS. 8B˜8G are 2D dot plots drawn by sampling the non-periodic signal of FIG. 8A with different time intervals;

FIG. 9 shows a flow chart of embodiment 2 of the present disclosure;

FIG. 10A˜10D show schematic diagram of the normal photoplethysmography (PPG) in accordance with embodiment 2 of the present disclosure;

FIG. 11A˜11D show schematic diagram of the abnormal PPG in accordance with embodiment 2 of the present disclosure;

FIG. 12A˜12D are 2D dot plot corresponding to FIG. 10A˜10D;

FIG. 13A˜13D are 2D dot plot corresponding to FIG. 11A˜11D;

FIG. 14 shows a block diagram of the exemplary hardware configuration in accordance with embodiment 3 of the present disclosure;

FIG. 15 shows a block diagram of the exemplary hardware configuration in accordance with embodiment 3A of the present disclosure;

### DETAILED DESCRIPTION OF THE INVENTION

The following description is made for the purpose of illustrating the general principles of the invention and should not be taken in a limiting sense.

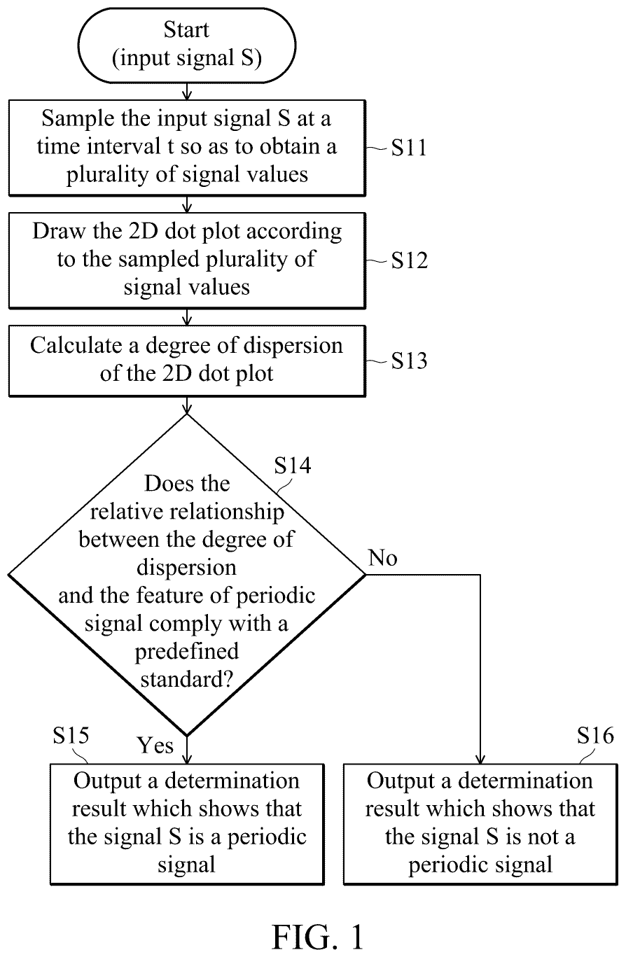

Please refer to FIGS. 1˜5, which describe embodiment 1 of the present disclosure. FIG. 1 shows a flow chart in accordance to embodiment 1 of the present disclosure. Embodiment 1 of the present disclosure is a method for determining the periodic signal. Steps of the method for determining the periodic signal can be implemented by an electronic circuit.

The electronic circuit may be microcontroller unit (MCU), single chip, single circuit, composite circuit, programmable processor, parallel programmable processor, IC logic integrated circuit (IC), gate array (GA), application specific integrated circuit (ASIC), or field programmable gate array (FPGA). The electronic circuit may also be central processing unit (CPU), digital signal processor (DSP), and graphics processing unit (GPU). In the following, the electronic circuit configured to implement each step of the method for determining whether a signal is periodic is also referred to as processing device.

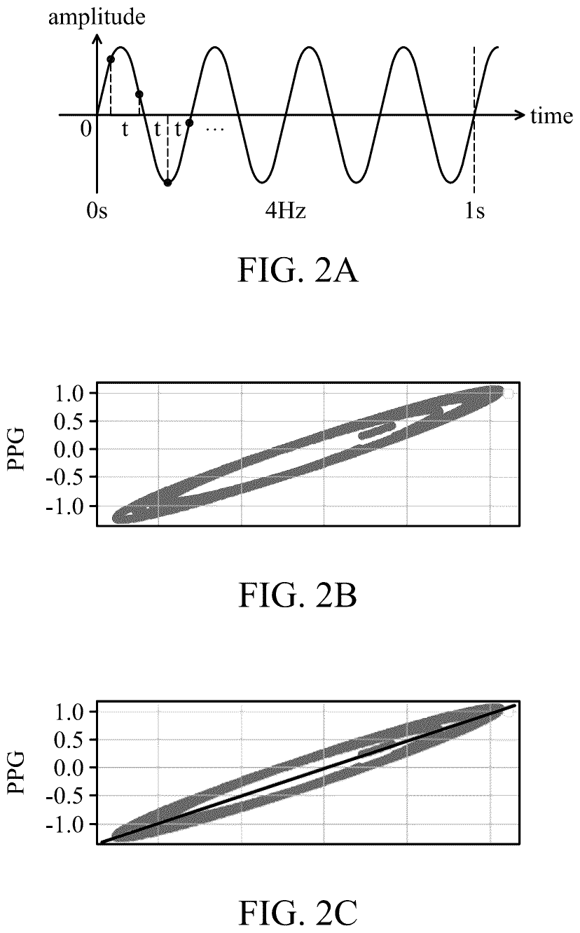

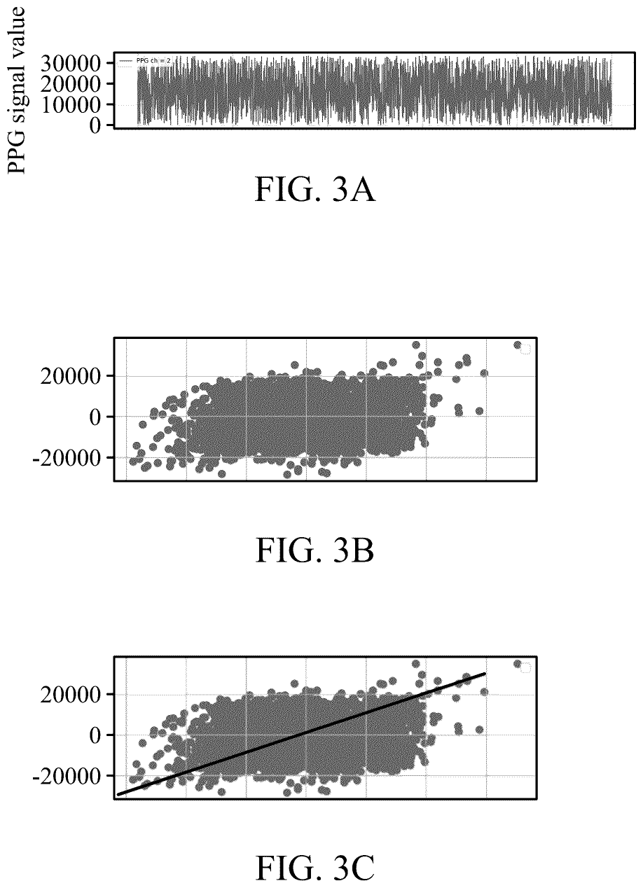

In the step S11, after a signal S input into a processing device, the processing device samples the input signal S at a time interval t so as to obtain a plurality of signal values. In some embodiment, time interval t is fixed. In the example of FIG. 2A, the signal S sampled by the processing device is a periodic signal P, and the processing device obtains a plurality of signal values P(t0+it). In the example of FIG. 3A, the signal S sampled by the processing device is a non-periodic signal N, and the processing device obtains a plurality of signal values N(t0+it). Wherein t0 is the first sample time, i is an integer ranging from 0 to total number of samples taken. The non-periodic signal N may be a random signal or a periodic signal containing a large amount of noises.

Fixed time interval t may be any time interval. The length of the time interval t will affect the shape of the 2D dot plot in the step S12. The details of the time interval t are described in the following paragraphs.

In the step S12, the processing device draws the 2D dot plot according to the plurality of signal values sampled in the step S11. The X coordinates of each point in the 2D dot plot is the sampled signal value, the Y coordinates of each point is the signal value obtained from the next sample.

For example, FIG. 2B is a 2D dot plot taking signal value P(t0+it) as the X coordinate and taking signal value P(t0+(i+1) t) as the Y coordinate. Due to the regular repetition of the signal value of the periodic signal P, the resulting 2D dot plot will have a narrow ellipse shape.

Furthermore, FIG. 3B is a 2D dot plot taking signal value N(t0+it) as the X coordinate (xi) and taking signal value N(t0+(i+1) t) as the Y coordinate (yi) ((xi,yi), i=0˜k, k is the total number of points on the 2D dot plot). Since the signal values of the non-periodic signal N aren't regular, the resulting 2D dot plot is randomly distributed.

The 2D dot plot may be generated by recording (drawing) the signal values as an array via the processing device. For example, a two-dimensional array Distr[X] [Y] may be constructed, which is initially a zero array. Each elements in the two-dimensional array Distr[X] [Y] is regarded as a point (x, y) on the XY plane. Then, the 2D dot plot can be drawn by changing the value of Distr[X] [Y] to 1 for each sampled signal value.

Then, in the step S13, the processing device calculates a degree of dispersion. In the embodiment 1, taking linear regression and least square method as example, an average error AE is calculated and regarded as the degree of dispersion.

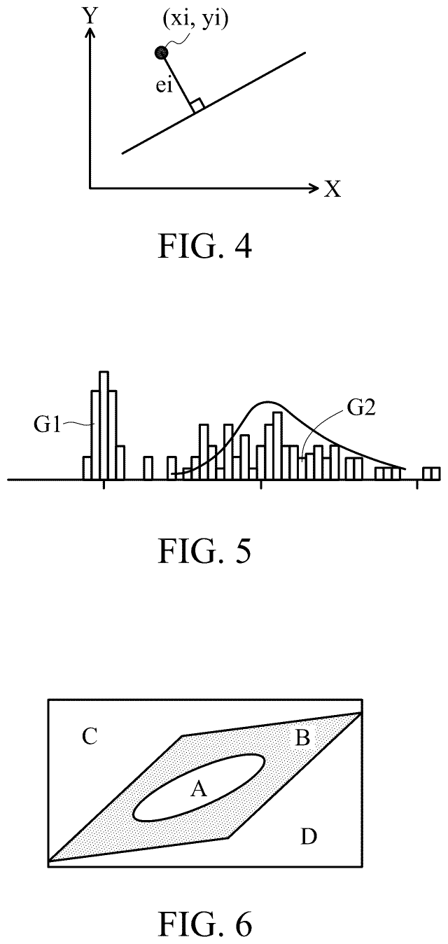

FIG. 2C shows the regression line of the 2D dot plot of FIG. 2B; and FIG. 3C shows the regression line of the 2D dot plot of FIG. 3B. Embodiment 1 calculates the average value (average error AE) of the distance from each point of the 2D dot plot projecting to the regression line, and the average value (average error AE) is regarded as the degree of dispersion. The distance projected to the regression line is shown in FIG. 4. The average error AE may be calculated by the following equation (1):

$$AE = \frac{{\sum }_{i = 1}^{k} ei}{k} \qquad ( 1 )$$

wherein k is the number of points on the 2D dot plot, and ei is the distance from each point projecting to the regression line.

After obtaining the average error regarded as the degree of dispersion, in the step S14, the processing device determines the relative relationship between the degree of dispersion and the feature of the periodic signal. Here, the feature of the periodic signal is a threshold Th relating to the degree of dispersion. In some embodiment, the processing device determines the relationship of the magnitudes between the degree of dispersion and the threshold Th, or compares the relationship between the degree of dispersion and the threshold Th, so as to determine the relative relationship between the degree of dispersion and the feature of the periodic signal. The processing device determines that the relative relationship between the degree of dispersion and the feature of the periodic signal complies with a predefined standard and thus determines that the signal S is periodic signal. The processing device determines that the relative relationship between the degree of dispersion and the feature of the periodic signal does not comply with the predefined standard and thus determines that the signal S isn't a periodic signal. In some embodiments, in response to the degree of dispersion being less than or equal to the threshold Th, the processing device determines that the relative relationship between the degree of dispersion and the feature of the periodic signal complies with the predefined standard. In response to the degree of dispersion being greater than the threshold Th, the processing device determines that the relative relationship between the degree of dispersion and the feature of the periodic signal does not comply with the predefined standard. If the processing device determines that the degree of dispersion is less than or equal to the threshold Th, which means that the points of the 2D plot fall within a specific range close to the regression line (as shown in FIG. 2C), the processing device outputs a determination result which shows that the signal S is a periodic signal (step S15). If the processing device determines that the degree of dispersion is larger than the threshold Th, which means that the points of the 2D plot do not fall within a specific range close to the regression line (as shown in FIG. 3C), the processing device outputs the determination result which shows that the signal S is not a periodic signal (step S15).

For example, methods for outputting the determination result in steps S15 and S16 may output text or voice based on the determination result via a display or a speaker. Alternatively, a signal configured to light up different color bulbs or to make the speaker produce a specific sound may be output in order to make known the determination result.

In some embodiments, the threshold Th may be predetermined and stored in a memory device so that the processing device can read the threshold Th. For example, the threshold may be determined in advance via experimentation or consultation of a database using the following method, and be stored in a non-volatile memory device.

For example, the method for determining the feature of the periodic signal (which is the threshold Th of the degree of dispersion in the embodiment 1) may be determining the threshold using cluster analysis, discriminant analysis, or principal component analysis. In some embodiments, the k-means algorithm is used to perform cluster analysis. Here, the k-means algorithm is taken as an example, but the present disclosure is not limited thereto. The threshold may be determined by linkage method, split method, hierarchical method, Gaussian mixture models (GMM), mean shift, and the like.

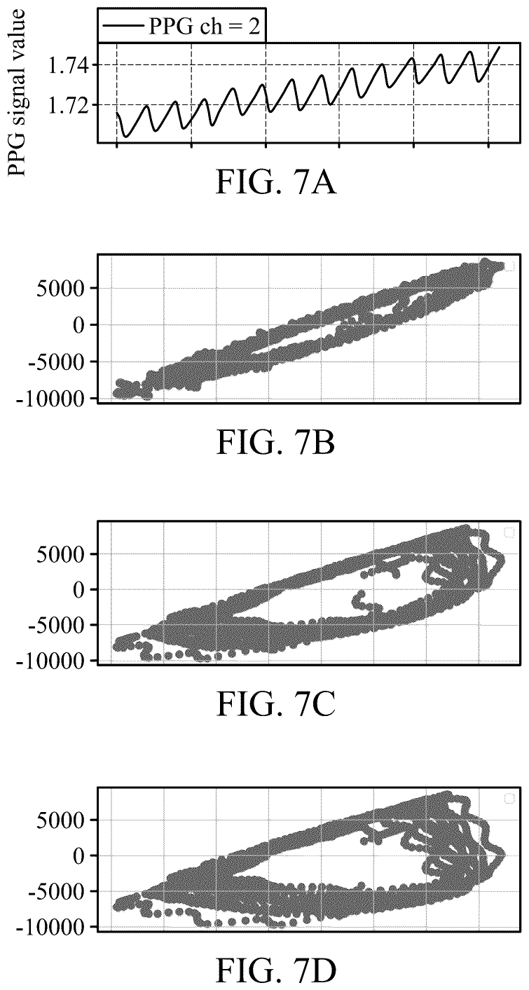

FIG. 5 shows the K-means method. In the K-means method, data of the average error AE obtained by performing steps S11˜S13 to a plurality of signals is plotted as a bar chart. The horizontal axis of the bar chart is the average error AE, and the vertical axis of the bar chart is the number of times the average error AE occurs. When there is enough data, two regions G1, G2 with Gaussian distribution will appear in the bar chart. The narrower region G1 is the Gaussian distribution of the data with smaller average error AE (periodic signal), and the wider region G2 is the Gaussian distribution of the data with larger average error AE (non-periodic signal). The value (the place where the arrow is pointing) between region G1 and region G2 is the optimal classification point and can be taken as the threshold.

As described above, the method for calculating the degree of dispersion and determining the feature of the periodic signals may be implemented by other techniques. In the following, embodiment 1A illustrates how to use a template diagram M to calculate the degree of dispersion.

In the embodiment 1A, the template diagram M is divided into A, B, C, and D four blocks. Each block of the template diagram M has a corresponding score, wherein A block is 1 point, B block is 5 point, C block is-10 point, and D block is-10 point. The template diagram M may be predetermined and stored in a memory device so that the processing device can read the template diagram M. For example, the template diagram M may be determined in advance via experimentation or consultation of a database, and be stored in a non-volatile memory device.

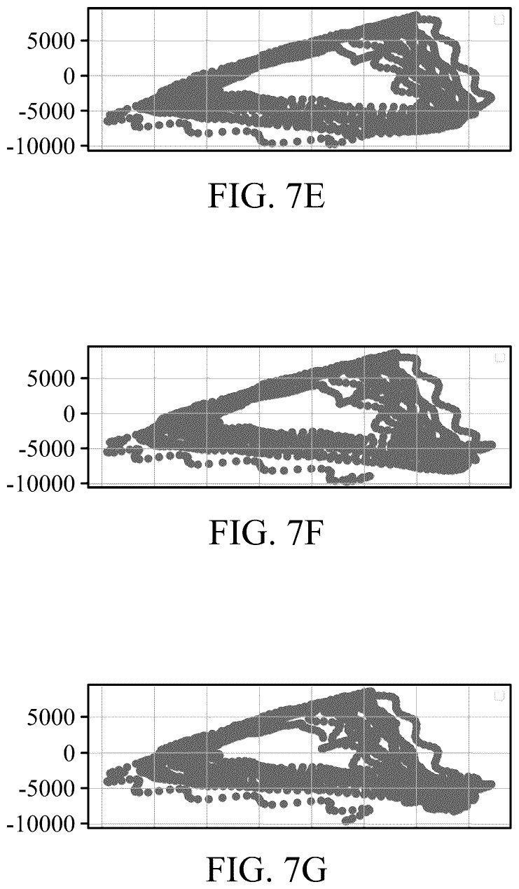

In the step S12, the processing device generates the 2D dot plot corresponding to the template diagram M, and the step S13 calculates the degree of dispersion by the template diagram M shown in FIG. 6. For example, in step S12, a 2D dot plot having the same size as the template diagram M is generated. In step S13, the 2D dot plot is compared with the template diagram M, the number of points in each block of the 2D dot plot corresponding to the template diagram M is multiplied by the score of the block, and the results of the number of points multiplied by the score are added up for each block, so as to obtain the total score of the 2D dot plot corresponding to the template diagram M. The total score is regarded as the degree of dispersion. Alternatively, the processing device determines the score of each point and summarizes the score, during drawing each point of the 2D dot plot in the step S12. The processing device calculates the total score and regard it as the degree of dispersion at the time step S12 is completed. In this embodiment, the higher the total score (which is regarded as the degree of dispersion of the signal), the closer the signal is to the periodic signal. Thus, a threshold Th may be set. Signals with a degree of dispersion that meet or exceed the threshold Th are determined to be periodic signals, and signals with a degree of dispersion that is under threshold Th are determined to be non-periodic signals.

Moreover, because the shape of the 2D dot plot will change according to the sample time interval t and type of signal, the template diagram M shown in FIG. 6 is just an example. The shape and point of the template diagram M can be adjusted according to the sample time interval t and type of signal. An example of adjusting the template diagram M according to the sample time interval t is described below.

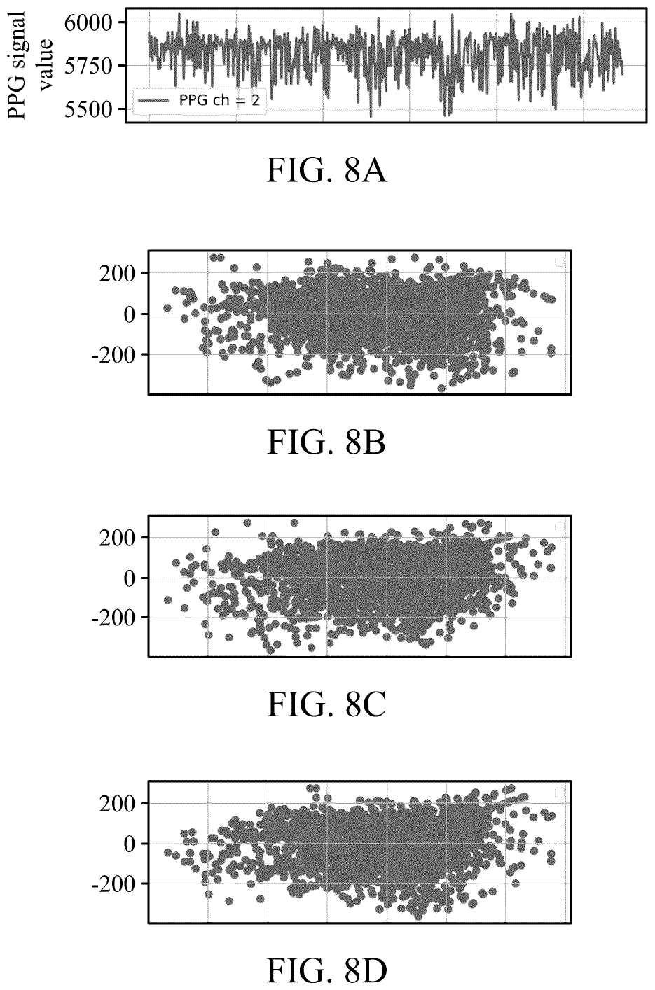

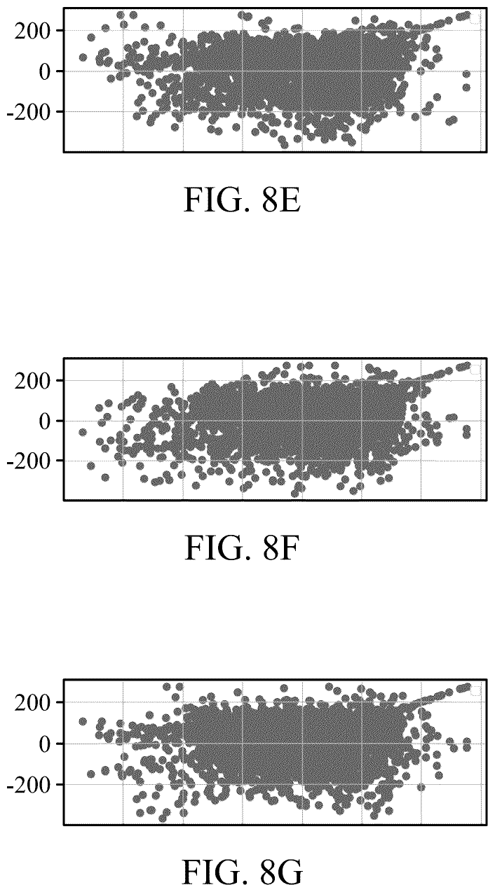

FIG. 7A shows a periodic signal. FIG. 7B˜7G are 2D dot plots drawn by sampling the periodic signal of FIG. 7A with different time intervals (7, 20, 30, 40, 50, 60 times per minute respectively). As shown in FIG. 7B˜7G, when the sampling time interval becomes shorter, since the signal values obtained between two samples are similar, the 2D dot plot will be in the shape of a slender line close to the x=y line. When the sampling time interval becomes longer, the distribution range of the dot plot becomes wider (e.g. hollow triangle shape in FIG. 7G). Furthermore, FIGS. 8B˜8G show that the non-periodic signal (FIG. 8A) sampled at different time intervals t. According to FIGS. 8B˜8G, it can be known that regardless of the length of the sampling interval t, the distribution range of the dot plot is extremely large. Thus, shape of the template diagram, score of the template diagram, and threshold configured to determine the periodic signal may be adjusted according to different sampling time intervals t.

Other steps of embodiment 1A are as described in embodiment 1 and are not repeated here.

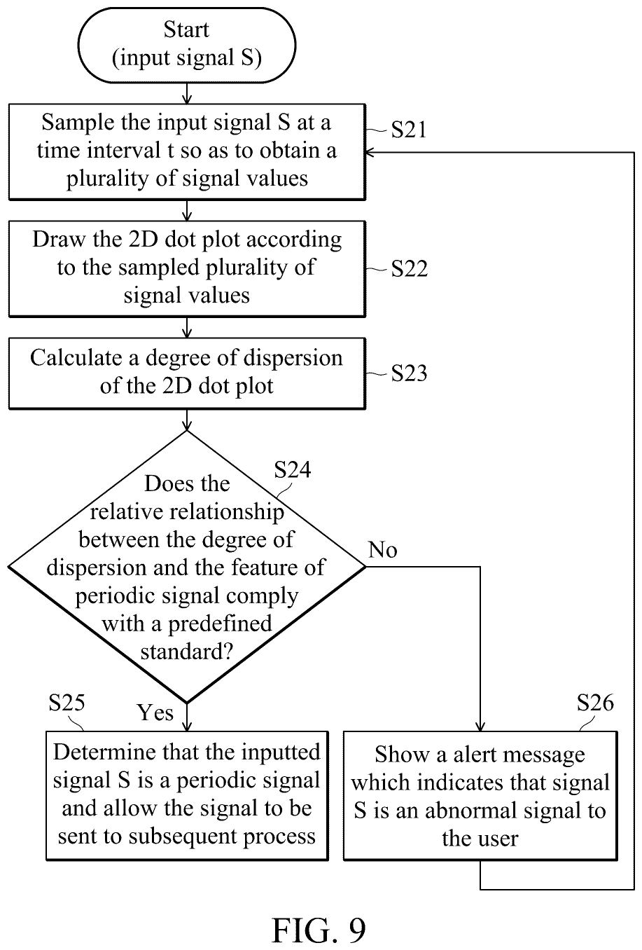

FIG. 9 shows a flow chart of embodiment 2. Embodiment 2 of the present disclosure is method for determining biomedical signals, in particular, a method for determining photoplethysmography (PPG) signals. Steps of the method for determining PPG signals may be implemented by electronic circuits.

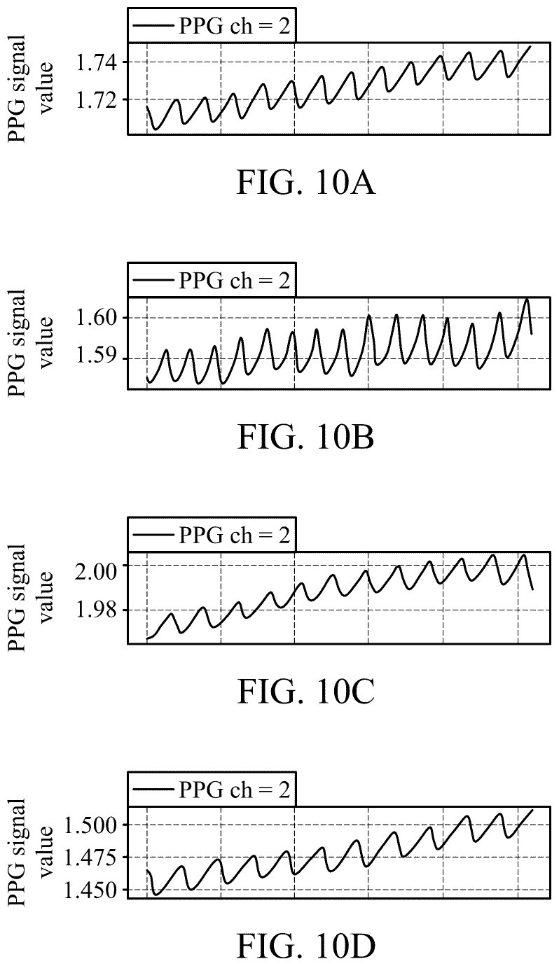

Most biomedical signals are characterized by cyclical changes, which means that most biomedical signals are periodic signals. Photoplethysmography (PPG) is a plethysmogram obtained by measuring with optical means. FIGS. 10A˜10D show normal PPG patterns of the finger placed in the sample cabin. The PPG patterns shown in FIGS. 10A˜10D are measured with optical means.

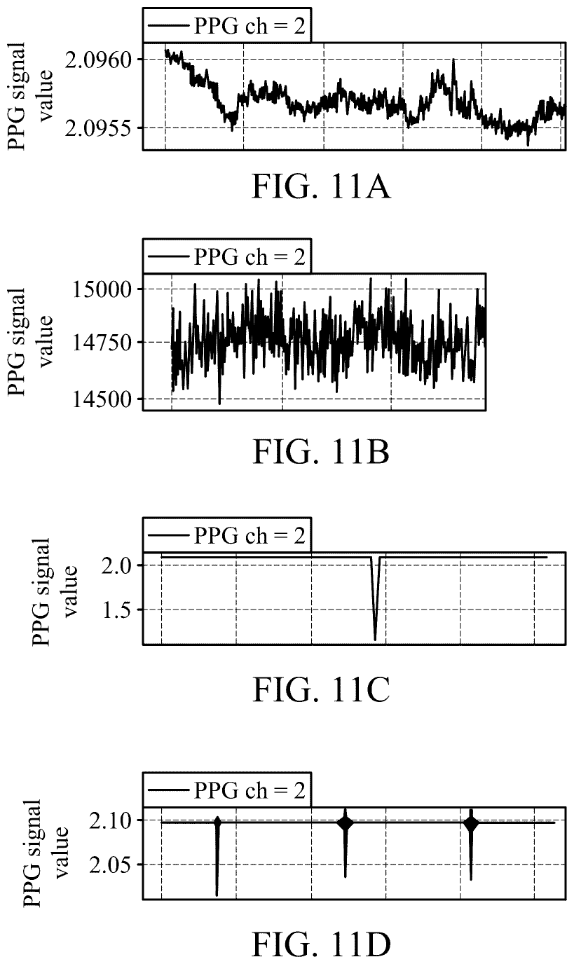

However, when measuring biomedical signals, abnormal signals containing large amount of noise are often obtained because of the unsatisfactory state of the measurement environment or the object under test. FIG. 11A shows abnormal PPG signal caused by excessively moist finger. FIG. 11B shows abnormal PPG signal caused by finger not placing in sample cabin. FIGS. 11C˜11D show abnormal PPG signals caused by excessive finger pressure.

To make the analysis results accurate and reliable, the biomedical signal determination method may be performed before analyzing the biomedical signals, so that the subsequent processing will only be performed on the biomedical signals determined as periodic signal. When a biomedical signal is determined as a non-periodic signal, the user is reminded to re-adjust the measurement environment. That is, embodiment 2 of the present disclosure may be used as a pre-processing of the processing of biomedical signals.

As shown in FIG. 9, steps S22˜S24 of embodiment 2 are the same as steps S12˜S14 of embodiment 1. Only step S21, step S25, and step S26 are described below.

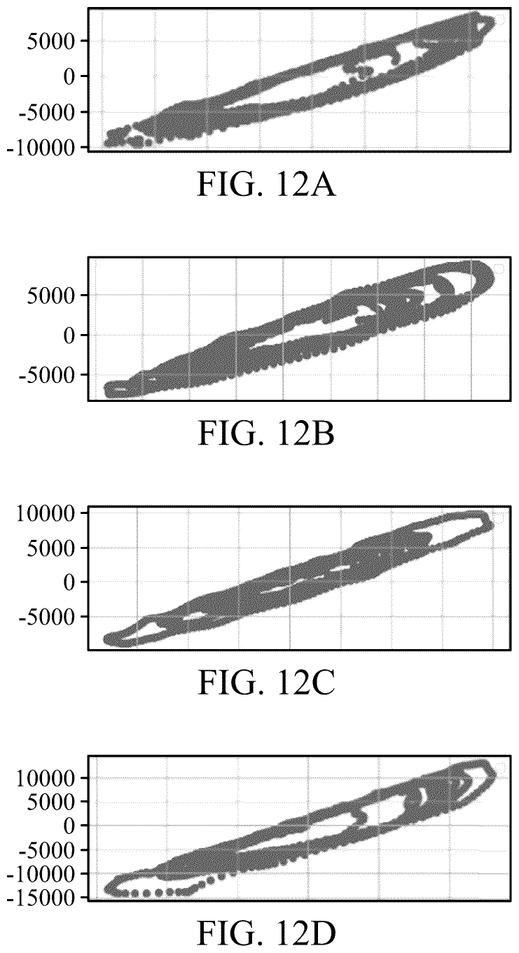

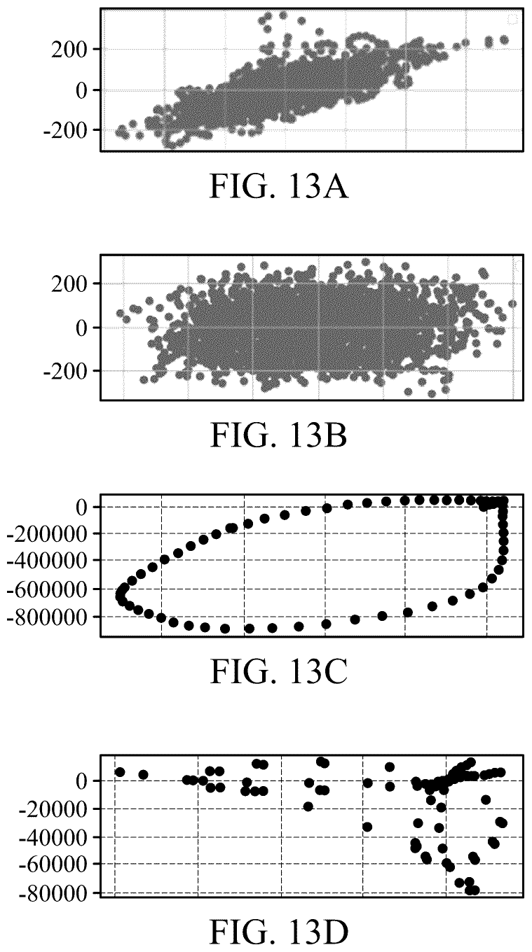

After a signal S is input into the processing device, in step S21, the processing device samples the signal S at a fixed time interval t. FIGS. 12A˜12D show examples of the processing device sampling normal signals at the fixed time interval t. FIGS. 13A˜13D show examples of the processing device sampling abnormal signals at the fixed time interval t.

In the present embodiment, the fixed time interval t may be determined by referring human heart rate (HR). For example, the fixed time interval t may be determined as the maximum integer less than one-tenth of the heart rate, i.e. [HR/10]. Take common heart rate 72 beats per minute (72 bpm) as example, [72/10]=7, i.e. sampling 7 times per minute, and the fixed time interval is 60/7 second. Alternatively, the fixed time interval t may be determined as the minimum integer not less than one-tenth of the heart rate, i.e. [HR/10], and the like. Moreover, fixed time interval t used to sample is not limited to integers. The fixed time interval t may be any time interval which can be used by the processing device to take samples.

In step S25, the processing device determines that the input signal S is a periodic signal. At this time, the processing device allows the signal to be sent to subsequent process. For example, the processing device allows the input signal S to be sent to other algorithm modules so as to perform signal processing such as physiological measurement feature acquisition, feature analysis, and/or deep learning. Furthermore, the processing device may also outputs a determination result, which indicates that signal S is a periodic signal, for the user to read, as described in embodiment 1.

In step S26, the processing device determines that the input signal S is a non-periodic signal. At this time, the processing device reminds the user that signal S is an abnormal signal and that the measurement environment is required to be adjusted and goes back to step S21 to sample the signal S. Method for reminding the user may be causing the display or the speaker to output words or voice based on the determination result. Alternatively, the method may output a signal that causes a bulb having a particular color to glow, or causing the speaker to generate a particular sound.

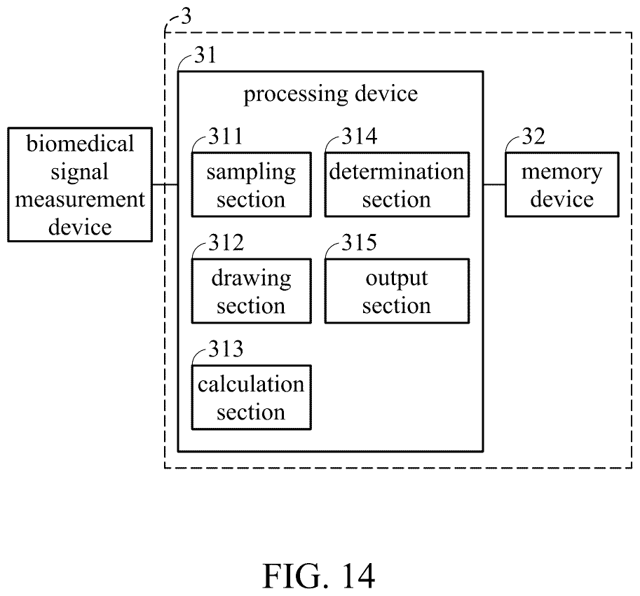

FIG. 14 shows a block diagram of the example hardware configuration in accordance to embodiment 3. Embodiment 3 is a photoplethysmography (PPG) signal analysis device 3, which includes sampling section 311, drawing section 312, calculation section 313, determination section 314, output section 315, and memory device 32. Each above mentioned section may be implemented as a program module performed by a processing device 31, and the program module may be stored in the memory device 32. Alternatively, all or a part of the above mentioned section may be implemented via electronic circuits.

In some embodiments, PPG signal analysis device 3 is the device which performs the biomedical signal determination method in embodiment 2. PPG signal analysis device 3 may be combined with other device configured to process the PPG signals. For example, PPG signal analysis device 3 may be combined with a wearable device, which can measure and analysis human PPG signals, so as to filter out the abnormal signals before the wearable device analyzes the human PPG signals and improve the accuracy of the subsequent PPG signal processing results. In some embodiments, PPG signal analysis device 3 is coupled to a biomedical signal measurement device in the wearable device and receives the PPG signal measured by the biomedical signal measurement device. For example, the biomedical signal measurement device may include light-emitting device and light-sensing device. The light-emitting device can emit light toward human body (e.g. finger, but not limited thereto), and the light-sensing device may be configured to receive the light reflected from the human body so as to generate corresponding signal as PPG signal. In an embodiment, the light-emitting device is light emitting diode, and the light-sensing device is photo diode. However, the present disclosure is not limited thereto.

When the PPG signal analysis device 3 receives the signal S input from external, sample section 311 samples the signal S at the fixed time interval to obtain a plurality of signal values S(t0+it).

As described in embodiment 2, the fixed time interval t may be determined by referring human heart rate (HR). In this embodiment, the fixed time interval t may also be set by the user using the PPG signal analysis device 3. For example, the fixed time interval t may be set according to the current heart rate of the user or according to the previous heart rate history record of the user.

The drawing section 312 takes the plurality of signal values S(t0+it) as X coordinates and takes the signal values S(t0+(i+1) t) for the next sample of the plurality of signal values as Y coordinates so as to generate the 2D dot plot.

The calculation section 313 calculates the degree of dispersion of the above mentioned 2D dot plot. The degree of dispersion may be calculated by any method. In this embodiment, the linear regression and least square method described in the embodiment 1 are used to calculate the average error AE between each point on the 2D dot plot and the regression line, and the average error AE is regarded as the degree of dispersion. Moreover, the template diagram described in the embodiment 1A may be used to the degree of dispersion.

The determination section 314 determines the relative relationship between the degree of dispersion and the feature of the periodic signals and generates the determination result. In this embodiment, as embodiments 1 and 1A, the threshold Th of the degree of dispersion is also regarded as the feature of the periodic signals. In some embodiments, the determination section 314 determines the relationship of the magnitudes between the degree of dispersion and the threshold Th so as to determine the relative relationship between the degree of dispersion and the feature of the periodic signals. The threshold Th may be stored in the memory device 32 in advance. As described in the embodiments 1 and 2, the threshold Th may be determined via consultation of a database or from experimental data.

The determination section 314 determines that the degree of dispersion is greater than the threshold Th and thus determines that the relative relationship between the degree of dispersion and the feature of the periodic signals does not comply with the predefined standard. In response to the determination result showing that the degree of dispersion is greater than the threshold Th, this means that the signal S is an abnormal signal containing noise, rather than a periodic signal. In response to the determination result indicating that the relative relationship between the degree of dispersion and the feature of the periodic signals does not comply with the predefined standard, i.e. the degree of dispersion is greater than the threshold Th, the output section 315 keeps outputting the alert message. The alert message reminds the user to re-adjust the measurement environment, until the determination result indicates that the relative relationship between the degree of dispersion and the feature of the periodic signals complies with the predefined standard (i.e. the degree of dispersion less than or equal to the threshold Th). Namely, the degree of dispersion of the 2D dot plot drawn according to the newly input signal S is less than or equal to the threshold Th. For example, the output section 315 keeps outputting the signal, which causes the display or the speaker on the PPG signal analysis device 3 to output words or voice, so as to remind the user to re-adjust the measurement environment until the newly input signal S is determined as a periodic signal by the output section 315. Alternatively, the output signal may cause a bulb, which has a particular color, on the PPG signal analysis device 3 to glow or cause the speaker on the PPG signal analysis device 3 to generate a particular sound so as to remind the user to re-adjust the measurement environment. Moreover, in response to the determination result indicating that the degree of dispersion is greater than the threshold Th, the processing device 31 may block the signal S to prevent the signal S from entering the subsequent PPG signal processing, which would generate an inaccurate result.

The determination section 314 determines that the degree of dispersion is less than the threshold Th and thus determines that the relative relationship between the degree of dispersion and the feature of the periodic signals complies with the predefined standard. In response to the determination result indicating that the degree of dispersion is less than the threshold Th, the signal S is a periodic signal, i.e. a normal PPG signal. At this time, the processing device 31 allows the signal S to undergo subsequent PPG signal processing.

In the above embodiments, as an alternative embodiment, a three-dimensional (3D) histogram may be drawn to replace the 2D dot plot. For example, a two-dimensional array Distr[X] [Y] may be built by the processing device, wherein X is the sampled signal value S(t0+it), and Y is the signal value S(t0+(i+1) t) for the next sample. In this way, the two-dimensional array Distr[X] [Y] may be regarded as the XY plane, and each element [X] [Y] in the array may be regarded as a point (x, y) on the XY plane. At this time, the number of occurrences of each element [X] [Y] may be calculated by calculating Distr[X] [Y]=Distr[X] [Y]+1 for each sampled signal value. That is, for each occurrence of the point (x,y), the height of the z-coordinate corresponding to that point is increased by 1.

When the signal S is a periodic signal, points on the XY plane will gather in the elliptical region near the line x=y. Thus, the 3D histogram will be higher. Conversely, when the signal S isn't a periodic signal, points on the XY plane will be distributed evenly. Thus, the 3D histogram will be lower in height. At this time, the height of the Z coordinate can be used to determine whether the signal S is a periodic signal. For example, the processing device may determine whether the maximum Z coordinate exceeds the threshold. Alternatively, the processing device may determine whether the average value of the Z coordinate exceeds the threshold.

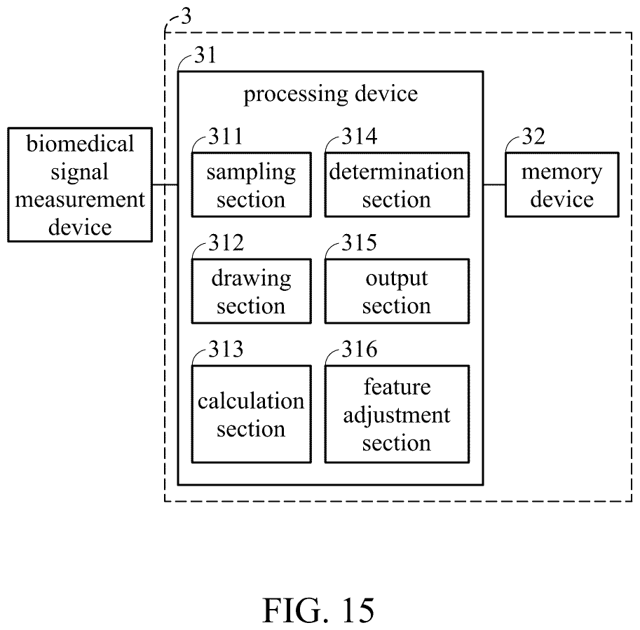

FIG. 15 shows a block diagram of the example hardware configuration in accordance to embodiment 3A. Embodiment 3A is a photoplethysmography (PPG) signal analysis device 3A that adds a feature adjustment section 316 to the PPG signal analysis device 3 of embodiment 3. Only the differences between embodiment 3A and embodiment 3 are described below.

Because the PPG signal analysis device 3A may be included in a personal wearable device owned by the user, in this embodiment, the PPG signal analysis device 3A may be adjusted by the user using the feature adjustment section 316. For example, the PPG signal analysis device 3A stores the degree of dispersion and the determination result in the memory section after each time the determination section 314 has made a decision. As described in the k-means method of embodiment 1, the threshold may be determined via consultation of a database or from experimental data in advance. In this embodiment, the threshold may be adjusted by redoing the k-means method using the feature adjustment section 316 after accumulating the user's personal database in the memory device 32.

According to the above description, the embodiments of the present disclosure can significantly reduce the amount of calculation, and thus it is possible to quickly determine whether the input signal is a periodic signal through simple calculation, and thereby reduce power consumption. Moreover, because the amount of calculation is smaller and there is no need to perform calculations using bulky hardware, the size of the device can be reduced and the power consumption can be reduced.

Although multiple embodiments have been described respectively, the embodiments may also be combined to implement. Alternatively, it is also possible to partially implement one of embodiment among multiple embodiments. Alternatively, it is also possible to partially combine multiple embodiments. Moreover, the configuration and steps described in the above embodiments may be changed in part as necessary.

The above embodiments are described for the purpose of making the invention more easy to understand, and the foregoing description is not intended to limit the present disclosure. Accordingly, the components disclosed in each of the above embodiments are intended to include all design variations or equivalents falling within the scope of the technology of the present disclosure.

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for determining whether a signal is periodic, performed by a processing device, comprising:
a coordinate-formation step, coordinating a signal S;
a degree-of-dispersion calculation step, calculating a degree of dispersion of a coordinate of the signal S; and
a determination step, determining a relative relationship between the degree of dispersion and a feature of a periodic signal and generating a determination result.

2. The method as claimed in claim 1, wherein:
the coordinate-formation step comprises:
sampling the signal S at a time interval t to obtain (k+1) signal values S (t0+i·t), wherein i=0˜k; and
generating a two-dimensional dot plot on an XY coordinate plane, wherein coordinates (x, y) of each point in the two-dimensional dot plot are (S(t0+i·t), S(t0+(i+1)·t)), wherein i=0˜(k−1);
the degree-of-dispersion calculation step comprises:
calculating the degree of dispersion of the two-dimensional dot plot.

3. The method as claimed in claim 2, wherein the degree of dispersion is calculated according to distances between each point in the two-dimensional dot plot and a linear regression line of each point in the two-dimensional dot plot.

4. The method as claimed in claim 2, wherein the degree of dispersion is calculated by calculating a score for each point in the two-dimensional dot plot in a template diagram.

5. The method as claimed in claim 4, wherein the feature of the periodic signal is determined using cluster analysis.

6. The method as claimed in claim 5, wherein the feature of the periodic signal is determined using a k-means algorithm.

7. A method for identifying photoplethysmography signal, performed by a processing device, comprising:
obtaining a photoplethysmography signal S_{PPG} from a photoplethysmography signal input device;
a coordinate-formation step, coordinating the photoplethysmography signal S_{PPG};
a degree-of-dispersion calculation step, calculating a degree of dispersion of coordinates of the photoplethysmography signal S_{PPG};
a determination step, determining a relative relationship between the degree of dispersion and a feature of a periodic signal and generating a determination result; and
determining that the relative relationship between the degree of dispersion and the feature of the periodic signal does not comply with a predefined standard, outputting an alert message and redoing the method for determining photoplethysmography signal.

8. The method as claimed in claim 7, wherein:
the coordinate-formation step comprises:
sampling the photoplethysmography signal S_{PPG} at a time interval t to obtain (k+1) signal values S_{PPG} (10+i·t), wherein i=0˜k; and
generating a two-dimensional dot plot on an XY coordinate plane, wherein the coordinates (x, y) of each point in the two-dimensional dot plot are (S_{PPG} (t0+i·t), S_{PPG} (t0+(i+1)·t)), wherein i=0˜(k−1);
the degree-of-dispersion calculation step comprises:
calculating the degree of dispersion of the two-dimensional dot plot.

9. The method as claimed in claim 8, wherein the degree of dispersion is calculated according to the distances between each point in the two-dimensional dot plot and a linear regression line of each point in the two-dimensional dot plot.

10. The method as claimed in claim 8, wherein the degree of dispersion is calculated by calculating the score of each point in the two-dimensional dot plot in a template diagram.

11. The method as claimed in claim 7, wherein the feature of the periodic signal is determined using a k-means algorithm.

12. The method as claimed in claim 11, wherein the time interval t is the maximum integer less than one-tenth of a heart rate.

13. The method as claimed in claim 11, wherein the feature of the periodic signal is determined again using the k-means algorithm according to the degree of dispersion and the determination result.

14. A photoplethysmography signal analysis device, comprising:
a photoplethysmography signal input device, configured to obtain a photoplethysmography signal S_{PPG};
a memory device, configured to store an application; and
a processing device, configured to perform the application so as to implement functions of the following sections:
a sampling section, configured to sample the photoplethysmography signal S_{PPG} at a time interval t to obtain (k+1) signal values S_{PPG} (t0+i·t), wherein i=0˜k;
a drawing section, configured to generate a two-dimensional dot plot on an XY coordinate plane, wherein the coordinates (x, y) of each point in the two-dimensional dot plot are (S_{PPG} (t0+i·t), S_{PPG} (t0+(i+1)·t)), wherein i=0˜(k−1);
a calculation section, configured to calculate a degree of dispersion of the two-dimensional dot plot;
a determination section, configured to determine a relative relationship between the degree of dispersion and a feature of a periodic signal and to generate a determination result; and
an output section, configured to output an alert message in response to the determination result indicating that the relative relationship between the degree of dispersion and the feature of the periodic signal does not comply with a predefined standard.

15. The device as claimed in claim 14, wherein the calculation section calculates the degree of dispersion according to the distances between each point in the two-dimensional dot plot and a linear regression line of each point in the two-dimensional dot plot.

16. The device as claimed in claim 14, wherein the calculation section calculates the degree of dispersion by calculating the score of each point in the two-dimensional dot plot in a template diagram.

17. The device as claimed in claim 14, wherein the feature of the periodic signal is determined using cluster analysis, and the feature of the periodic signal is stored in the memory section.

18. The device as claimed in claim 17, wherein the feature of the periodic signal is determined using a k-means algorithm.

19. The device as claimed in claim 17, wherein the time interval t is the maximum integer less than one-tenth of the heart rate.

20. The device as claimed in claim 17, wherein:
after the determination section has made each determination, the determination section stores the degree of dispersion and the determination result in the memory section;
the photoplethysmography signal analysis device further comprises:
a feature adjustment section, configured to determine the feature of the periodic signal again using the cluster analysis according to the degree of dispersion and the determination result stored in the memory section.
